(19) United States
(12) Patent Application Publication
(10) Pub. No.: US 2011/0071595 A1
(43) Pub. Date: Mar. 24, 2011

(54) SYSTEM FOR USING ELECTRICAL MUSCLE STIMULATION TO INCREASE BLOOD FLOW IN BODY PARTS

(76) Inventor: Philip Muccio, YPSILANTI, MI (US)

(21) Appl. No.: 12/564,542

(22) Filed: Sep. 22, 2009

(51) Int. Cl. A61N 1/36

(52) U.S. Cl. 607/48

(57) ABSTRACT

A system for stimulating muscles and increasing blood flow in one or more specific body parts is provided. This system includes at least one electrode adapted to be placed circumferentially around a body part, wherein the at least one electrode is operative to deliver electrical impulses sufficient to induce muscle contractions in the body part; at least one stimulation module in electrical communication with the at least one electrode, wherein the at least one stimulation module is operative to provide electrical impulses to the at least one electrode; and a control module in electrical communication with the at least one stimulation module, wherein the at least one control module is operative to control the characteristics of the electrical impulses.

### CROSS-REFERENCE TO RELATED APPLICATIONS

This patent application claims the benefit of U.S. Provisional Patent Application Ser. No. 61/225,703 filed on Jul. 15, 2009 and entitled “Electrical Stimulation System for Treatment of Deep Vein Thrombosis” and U.S. Non-Provisional patent application Ser. No. 11/466,637 filed on Aug. 23, 2006 and entitled “System and Device for Neuromuscular Stimulation,” the disclosures of which are incorporated by reference herein in their entirety and made part of the present U.S. non-provisional patent application for all purposes.

### BACKGROUND OF THE INVENTION

The described invention relates in general to a system for increasing blood flow in certain body areas of a patient, and more specifically to an apparatus and method for increasing blood flow in the trunk and extremities for medical and non-medical purposes using electrical muscle stimulation, also referred to herein as neuromuscular stimulation.

Diminished circulation in the extremities of a patient can lead to serious medical complications including death. Promoting circulatory health is essential for many patients who are susceptible to ongoing reduced blood or lymph flow stemming from pathology of the circulatory system, lymphatic system, or muscles (including paralysis from neurological insult); or in patients with short-term reduction in blood flow resulting from temporary incapacitation or immobilization, particularly from surgery involving the abdomen or joint replacement of the lower extremities. The incidence of deep vein thrombosis (DVT) in hip and knee arthroplasty typically ranges from forty to seventy percent. The incidence of pulmonary emboli (PE) in knee and hip arthroplasty patients is typically two to three percent. The causative factors surrounding the precipitation of DVT are known and include vessel wall and valvular damage and blood stasis resulting from immobilization during or following surgery. Removal of endogenous fibrinolysis which is produced by contracting muscles may also be a causative factor.

Muscle contractions are an integral component of the physiology of circulation. Without muscles of the lower extremity squeezing and pumping regularly, blood cannot efficiently return to the heart. Furthermore, when lower extremity muscles do not contract, there is little demand for oxygen, so the vascular system down regulates and directs the flow of blood away from the area. Thus, a complex series of mechanisms designed to facilitate both arterial and venal blood flow is diminished. Muscle contractions are also known to induce fibrinolysis, which can inhibit the formation of DVT.

Current approaches to DVT prophylaxis include a wide range of anti-coagulants and sequential compression devices (SCDs). SCDs are typically used as an adjunct therapy to facilitate blood flow to the lower extremities. However, a recent study comparing electrical stimulation to SCDs during total hip and knee arthroplasty to maintain or increase blood flow during the procedure clearly demonstrated that electrical stimulation is superior to SCDs. While cardiac output dropped 20% below baseline, the electrical stimulation group increased 20% (Faghri, IEEE 591: 62-69; March 1997).

Neuromuscular stimulation (NMES) is a technique which induces muscles to contract and produce increased blood flow. Involvement of the recipient of NMES in the activation of their muscles is unnecessary because NMES produces muscle activation independent of the recipient's involvement in the physiologic process. This involuntary effect is particularly advantageous for (i) surgical patients who are anesthetized or under a local epidural block; (ii) patients who are bedridden due to disability, age, or disease; or (iii) are paralyzed from spinal cord injury, stroke, or other neurological injuries or diseases. This effect would also be useful for people who are prevented from moving for long periods of time, such as those on long-duration flights or during extended period of sleep.

Despite the benefits of NMES, traditional electrical stimulation can sometimes be problematic with regard to the application of electrodes. Individual electrodes must be accurately placed over key motor points to elicit a proper muscle contraction. When electrodes are not placed properly, muscle recruitment causes unwanted movement, joint deviation, and heightened pain and discomfort from the stimulus, or it may not stimulate the muscle at all. Due to the level of skill required in properly placing the electrodes and the number of electrodes typically required for traditional NMES, the broad use of muscle stimulation for DVT prophylaxis in hospitals, for example, is currently impractical. Furthermore, electrical stimulation of muscle in its traditional form often causes discomfort to patients with intact sensation when the level of impulse is high enough to induce mild to moderate muscle contractions. Thus, there is an ongoing need for a system and method for using NMES to increase blood flow to certain regions or areas of the body, particularly the extremities that is relatively easy to implement and that does not cause pain or discomfort to the patient/recipient.

### SUMMARY OF THE INVENTION

The following provides a summary of certain exemplary embodiments of the present invention. This summary is not an extensive overview and is not intended to identify key or critical aspects or elements of the present invention or to delineate its scope.

In accordance with one aspect of the present invention, a system for electrically stimulating muscles and increasing blood flow in one or more body parts is provided. This system includes at least one electrode adapted to be placed circumferentially (i.e., completely) around a body part such as an arm or leg wherein the at least one electrode is operative to deliver electrical impulses sufficient to induce muscle contractions in the body part; at least one stimulation module in electrical communication with the at least one electrode, wherein the at least one stimulation module is operative to provide electrical impulses to the at least one electrode; and at least one control module in electrical communication with the at least one stimulation module, wherein the at least one control module is operative to control the characteristics of the electrical impulses.

In accordance with another aspect of the present invention, a system for electrically stimulating muscles and increasing blood flow to a body part is provided. This system includes at least two electrodes adapted to be placed circumferentially around a body part, wherein each of the at least two electrodes is operative to deliver electrical impulses sufficient to induce muscle contractions in the body part; at least one stimulation module in electrical communication with each of the at least two electrodes, wherein the at least one stimulation module is operative to provide electrical impulses to each of the at least two electrodes; and at least one control module in electrical communication with the at least one stimulation module, wherein the at least one control module is operative to control the characteristics of the electrical impulses.

In yet another aspect of this invention, a device for electrically stimulating muscles or muscle groups is provided. This device includes at least one electrode adapted to be placed circumferentially (i.e., completely) around a body part such as an arm, leg, or trunk, wherein the at least one electrode is operative to deliver electrical impulses sufficient to induce muscle contractions in the body part.

Additional features and aspects of the present invention will become apparent to those of ordinary skill in the art upon reading and understanding the following detailed description of the exemplary embodiments. As will be appreciated by the skilled artisan, further embodiments of the invention are possible without departing from the scope and spirit of the invention. Accordingly, the drawings and associated descriptions are to be regarded as illustrative and not restrictive in nature.

### BRIEF DESCRIPTION OF THE DRAWINGS

The accompanying drawings, which are incorporated into and form a part of the specification, schematically illustrate one or more exemplary embodiments of the invention and, together with the general description given above and detailed description given below, serve to explain the principles of the invention, and wherein:

FIG. 1 depicts an exemplary embodiment of the electrical muscle stimulation system of the present invention wherein multiple electrical stimulation assemblies have been placed on multiple body parts of an individual;

FIG. 2 illustrates the placement of an exemplary embodiment of the electrical stimulation system of the present invention wherein a single electrical stimulation assembly has been placed on the lower portion of one leg of an individual;

FIG. 3 is a cross-sectional view of a limb segment depicting the flow of an electrical current into both agonist and antagonist muscle groups from the circumferential electrode of the present invention;

FIG. 4 is a longitudinal view of a limb segment depicting the flow of an electrical current generated by the circumferential electrodes of the present invention; and

FIG. 5 is a graph depicting an exemplary order of stimulation for pushing blood proximally using the electrical muscle stimulation system of the present invention.

### DETAILED DESCRIPTION OF THE INVENTION

Exemplary embodiments of the present invention are now described with reference to the Figures. Reference numerals are used throughout the detailed description to refer to the various elements and structures. In other instances, well-known structures and devices are shown in block diagram form for purposes of simplifying the description. Although the following detailed description contains many specifics for the purposes of illustration, a person of ordinary skill in the art will appreciate that many variations and alterations to the following details are within the scope of the invention. Accordingly, the following embodiments of the invention are set forth without any loss of generality to, and without imposing limitations upon, the claimed invention.

The present invention relates to a neuromuscular stimulation system (NMES) for use in treating DVT and other medical and non-medical conditions by inducing singular and or co-contractions of muscle groups to increase blood and oxygen flow in those muscles. Other intended uses and benefits of the present invention include wound healing, muscle strengthening, and stimulation of bone growth. As previously indicated, first and second general embodiments of this invention provide a system for electrically stimulating muscles and increasing blood flow in one or more body parts such as the trunk or extremities; and a third general embodiment of this invention provides a device in the form of a circumferential electrode for electrically stimulating muscles or muscle groups, wherein the electrode is capable of completely encircling a human body part for increasing blood circulation. The electrode is supplied with electrical impulses to induce muscle contractions and may be shifted proximally or distally on the body part without degradation of muscle contraction while providing for concurrent agonist and antagonist muscle regeneration. With reference now to the Figures, one or more specific embodiments of this invention shall be described in greater detail.

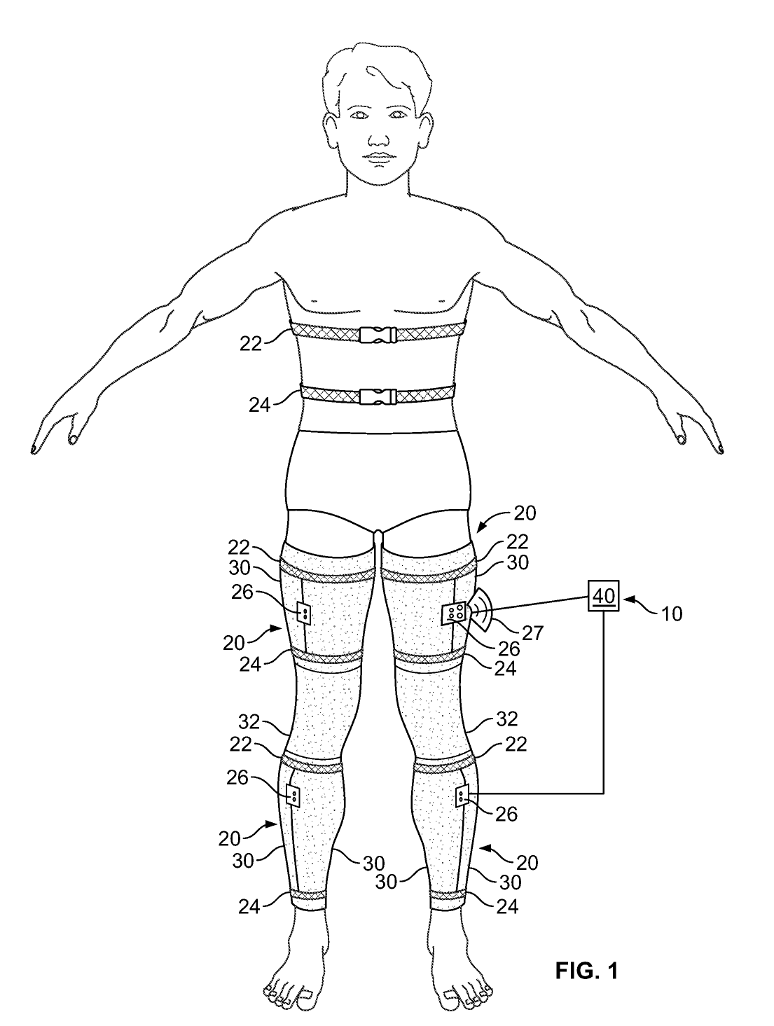

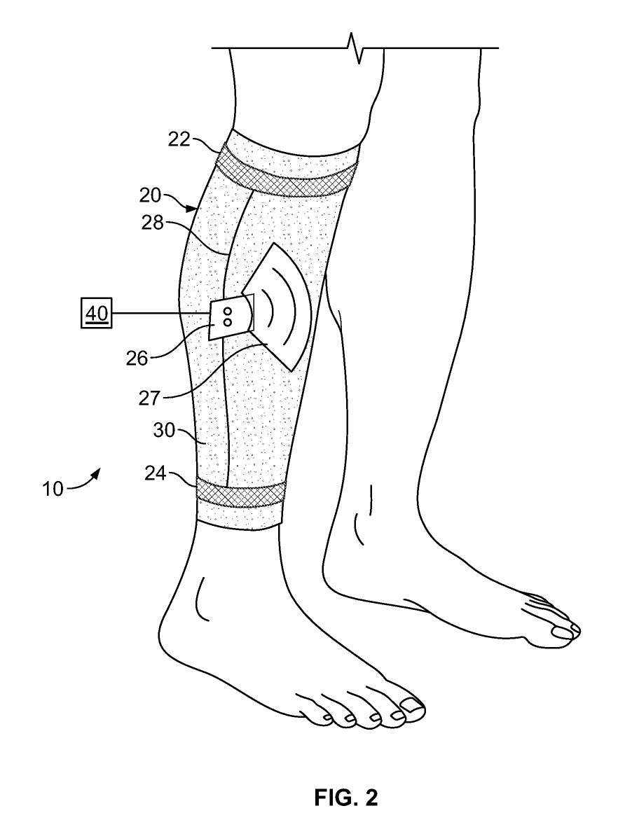

FIG. 1 depicts an exemplary embodiment of neuromuscular stimulation system 10, wherein multiple electrical stimulation assemblies 20 (or individual electrodes 22 ad 24) have been placed on multiple body parts of an individual. Each assembly 20 typically includes a first circumferential electrode 22 and a second circumferential electrode 24, both of which are in electrical communication with a stimulation module 26, which is connected to each circumferential electrode by at least one wire 28 (see FIG. 2). Stimulation module 26 typically communicates with control module 40 by wireless means, as depicted by reference number 27. Each assembly 20 typically includes a fabric sleeve 30, which acts as a substrate or support for the components thereof. One or more sleeves 30 may be included in a larger garment-like item 32, which may connect sleeves 30 to one another for the purpose a facilitating proper placement of system 10 on a user thereof. FIG. 2 illustrates the placement of an assembly 20 on the lower portion of one leg of an individual.

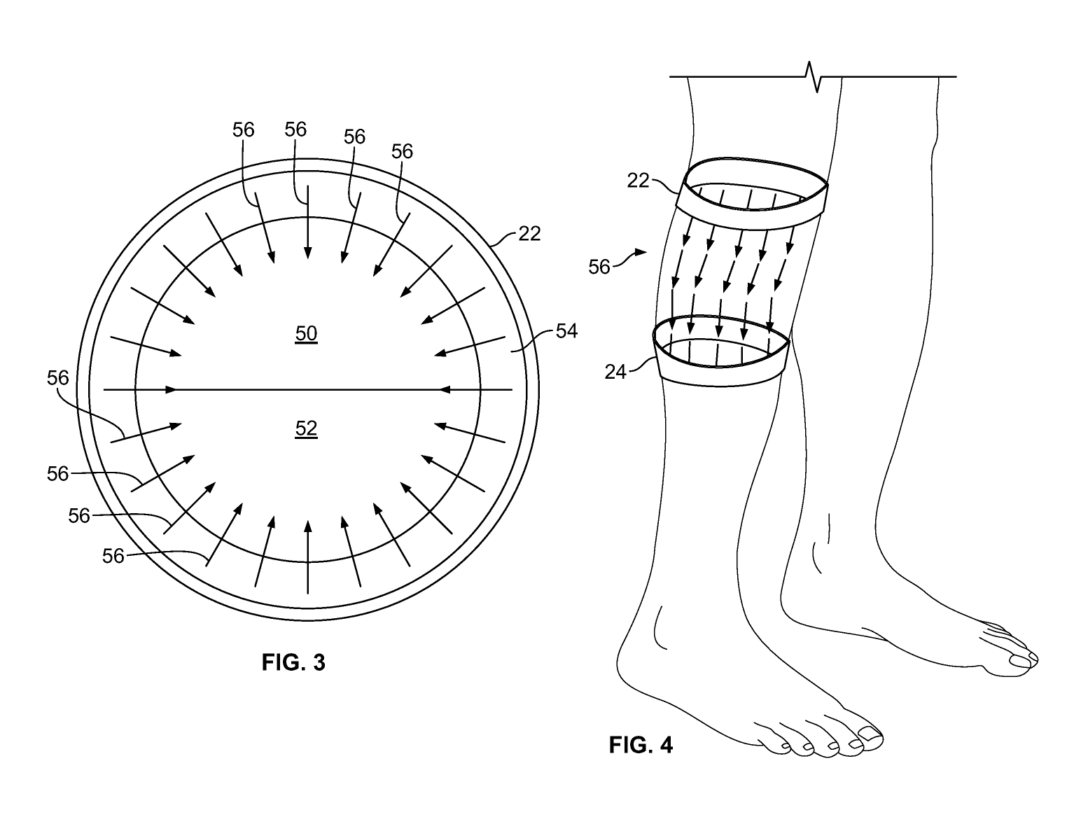

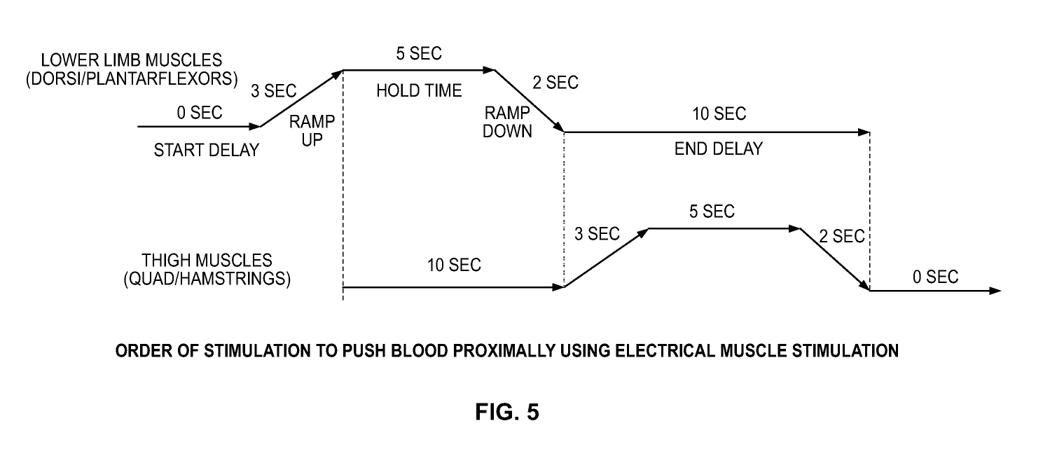

FIG. 3 provides a cross-sectional view of a limb segment depicting the flow of an electrical current into both agonist muscle 50 and antagonist muscle 52 from circumferential electrode 22. An electrical field 56 is generated by electrode 22 and passes through skin 54 into the muscles of the limb upon which electrode 22 has been placed. FIG. 4 is a longitudinal view of a limb segment depicting the flow of an electrical current generated by the circumferential electrodes of the present invention. In this Figure, electrodes 22 and 24 have been placed in sequence (i.e., tandem) on the leg of a patient for the purpose of creating electrical field 56. This electrical field is operative to push blood proximally in the leg and increase circulation therein by inducing a co-contraction (simultaneous contraction) of agonist and antagonist muscles. FIG. 5 provides a graph that depicts an exemplary method of electrical stimulation for pushing blood proximally using an exemplary embodiment of neuromuscular stimulation system 10.

When system 10 is in use, each muscle group (agonist and antagonist) being treated is typically controlled by one set of circumferential electrodes 22, 24 and one integrated stimulation module 26, which coordinates the electrical impulses between the electrodes, which may be controlled remotely from the electrodes. As will be appreciated by one of ordinary skill in the art, stimulation module 26 is equipped with electronics and battery power used deliver adequate stimulation. Each stimulation module 26 receives a wireless signal 27 from control module 40 for properly coordinating the contraction of muscle groups. Stimulation module 26 may also include electronic means for monitoring contractions of a body part and providing selected electrical impulses to the circumferential electrodes. In an exemplary embodiment, stimulation module 26 has a width of approximately two inches or less and may be extensible in length.

In an exemplary embodiment, the sequence of muscle contraction is first the dorsiflexors and plantarflexors (triggered simultaneously), then as those muscles begin to relax, the quadriceps and hamstrings (triggered simultaneously) are stimulated (see FIG. 5). This produces a natural push of blood cephalad. Through the use of known technology, control module 40 is also capable of monitoring the status of equipment, electrode impedance, compliance and blood flow and then delivering this information to clinicians. Control module 40 may be used to adjust the level of electrical stimulation in accordance with the need for increased or decreased blood flow in or out of the treated limb in a closed-loop feedback manner.

The circumferential electrodes of the present invention are typically 1-2 inches in width and the area of coverage is significantly higher than the conventional electrodes typically used in NMES. Thus, the current density (CD) of the circumferential electrodes is significantly lower, even at high intensity outputs, meaning they are more comfortable than conventional electrodes. For example a conventional electrode would have an area of 2×4 inches equaling 8 square inches; whereas a circumferential electrode (around a typical thigh) would have an area 1×25 inches equaling 25 square inches. With a conventional electrode, a current density of 100 mA equals 100 mA per 8 square inches or 12.5 mA per square inch; whereas with a circumferential electrode, a current density of 100 mA equals 100 mA per 25 square inches or 4 mA per square inch. In an exemplary embodiment, the electrical impulse parameters for each circumferential electrode include an amplitude of about 0.1-150 mA; a pulse width of about 0.1-400 microseconds; a frequency of 25-50 Hz; and a duty cycle of about 5-10 seconds ON, and about 10-60 seconds OFF.

In certain embodiments of this invention, circumferential electrodes 22, 24 utilize either an adhesive or non-adhesive biogel to interface with the patient's skin. This biogel may include hydrogel polymers, polymerized polyethylene glycol diacrylate, polylactic acid, polyglycolic acid, polymerized polyethylene glycol dimethylacrylate and mixtures thereof. The circumferential electrodes of the present invention are extensible in length (including the biogel) to accommodate expansion in girth as muscle contracts so as to not constrict the limbs and to maintain electrode-to-skin contact; and are flexible for accommodating the contours of limbs which may vary widely due to variations in underlying muscle and bone. Neuromuscular stimulation system 10 and the various components thereof may be provided as a portable kit that includes instructions for proper placement of the electrodes and proper use of the system.

A primary advantage of the present invention is that circumferential electrodes 22, 24 transmit electrical current through the patient's skin and body in a highly efficient manner. The electrical current generated by these circumferential electrodes travels in a perpendicular fashion with respect to the planar surface of the electrode and then along a parallel pathway, in the long direction of the limb segment (see FIGS. 3-4). These aspects of the disclosed circumferential electrodes results in much less discomfort to the patient. The reduced level of discomfort is also due to lower current densities emitted by the circumferential electrodes as compared to more traditional electrodes. Furthermore, the circumferential electrodes of the present invention do not have to be placed accurately to effectively excite muscle tissue. These electrodes may be shifted proximally or distally, while still inducing muscle contractions without compromising the quality of stimulation or level of comfort. Finally, as previously discussed, the disclosed circumferential electrodes reduce the number of required electrodes and stimulation channels by one-half, making a NMES system utilizing these electrodes far easier to operate than known systems.

While the present invention has been illustrated by the description of exemplary embodiments thereof, and while the embodiments have been described in certain detail, it is not the intention of the Applicant to restrict or in any way limit the scope of the appended claims to such detail. Additional advantages and modifications will readily appear to those skilled in the art. Therefore, the invention in its broader aspects is not limited to any of the specific details, representative devices and methods, and/or illustrative examples shown and described. Accordingly, departures may be made from such details without departing from the spirit or scope of the Applicant's general inventive concept.

What is claimed:

1) A system for stimulating muscles and increasing blood flow, comprising:
(a) at least one electrode adapted to be placed circumferentially around a body part, wherein the at least one electrode is operative to deliver electrical impulses sufficient to induce muscle contractions in the body part;
(b) at least one stimulation module in electrical communication with the at least one electrode, wherein the at least one stimulation module is operative to provide electrical impulses to the at least one electrode; and
(c) a control module in electrical communication with the at least one stimulation module, wherein the at least one control module is operative to control the characteristics of the electrical impulses.

2) The system of claim 1, further comprising a biogel for providing an interface between the electrode and the body part, and wherein the biogel is adhesive or non-adhesive.

3) The system of claim 2, wherein the biogel is a hydrogel polymer, polymerized polyethylene glycol diacrylate, polylactic acid, polyglycolic acid, polymerized polyethylene glycol dimethylacrylate, or combinations thereof.

4) The system of claim 1, wherein the electrode is about 1-2 inches in width.

5) The system of claim 1, wherein the electrode is extensible in length.

6) The system of claim 1, wherein each electrical impulse has an amplitude of from about 0.01 to 150 mA, a pulse width of from about 0.01 to 400 microseconds, a frequency of from about 25 to 50 Hz, and a duty cycle of from about of from 5 to 10 seconds on and from 10 to 60 seconds off.

7) The system of claim 1, wherein the control module is further operative to monitor the muscle contractions and adjust the electrical impulses to increase or decrease blood flow in the body part.

8) A system for stimulating muscles and increasing blood flow, comprising:
(a) at least two electrodes adapted to be placed circumferentially around a body part, wherein each of the at least two electrodes is operative to deliver electrical impulses sufficient to induce muscle contractions in the body part;
(b) at least one stimulation module in electrical communication with each of the at least two electrodes, wherein the at least one stimulation module is operative to provide electrical impulses to each of the at least two electrodes; and
(c) at least one control module in electrical communication with the at least one stimulation module, wherein the at least one control module is operative to control the characteristics of the electrical impulses.

9) The system of claim 8, further comprising a biogel for providing an interface between the electrodes and the body part, and wherein the biogel is adhesive or non-adhesive.

10) The system of claim 9, wherein the biogel is a hydrogel polymer, polymerized polyethylene glycol diacrylate, polylactic acid, polyglycolic acid, polymerized polyethylene glycol dimethylacrylate, or combinations thereof.

11) The system of claim 8, wherein the electrodes are about 1-2 inches in width and are extensible in length.

12) The system of claim 8, wherein the electrodes are operative to treat DVT, affect wound healing, increase muscle strength, or stimulate bone growth.

13) The system of claim 8, wherein each electrical impulse has an amplitude of from about 0.01 to 150 mA, a pulse width of from about 0.01 to 400 microseconds, a frequency of from about 25 to 50 Hz, and a duty cycle of from about of from 5 to 10 seconds on and from 10 to 60 seconds off.

14) The system of claim 8, wherein the control module is further operative to monitor the muscle contractions and adjust the electrical impulses to increase or decrease blood flow in the body part.

15) A device for electrically stimulating muscles, comprising: at least one electrode adapted to be placed circumferentially around a body part, wherein the at least one electrode is operative to deliver electrical impulses sufficient to induce muscle contractions in the body part.

16) The device of claim 15, wherein the at least one electrode is adapted to communicate with at least one stimulation module, and wherein the at least one stimulation module is operative to provide electrical impulses to the at least one electrode.

17) The device of claim 16, wherein each electrical impulse has an amplitude of from about 0.01 to 150 mA, a pulse width of from about 0.01 to 400 microseconds, a frequency of from about 25 to 50 Hz, and a duty cycle of from about of from 5 to 10 seconds on and from 10 to 60 seconds off.

18) The device of claim 15, wherein the stimulation module is adapted to communicate with at least one control module, and wherein the at least one control module is operative to control the characteristics of the electrical impulses.

19) The device of claim 15, wherein the electrode is about 1-2 inches in width and is extensible in length.

20) The device of claim 15, wherein the electrode is operative to treat DVT, affect wound healing, increase muscle strength, or stimulate bone growth.
